(19) United States
(12) Patent Application Publication
(10) Pub. No.: US 2019/0390153 A1
(43) Pub. Date: Dec. 26, 2019

(54) FULLY AUTOMATIC CELL CULTURE METHOD AND SYSTEM THEREOF BASED ON MECHANICAL ARM

(71) Applicant: Cellauto Biological Automation CO., LTD, Shenzhen (CN)

(72) Inventors: Xiang Hu, Shenzhen (CN); Muyun Liu, Shenzhen (CN)

(21) Appl. No.: 16/527,008

(22) Filed: Jul. 30, 2019

Related U.S. Application Data
(63) Continuation of application No. PCT/CN2018/096105, filed on Jul. 18, 2018.

(30) Foreign Application Priority Data
Jun. 26, 2018 (CN) 201810666385.3

(51) Int. Cl. C12M 1/34; C12M 1/36

(52) U.S. Cl. CPC C12M 41/32; C12M 41/16; C12M 41/48

(57) ABSTRACT

A fully automatic cell culture method and system thereof based on mechanical arm are disclosed, and the method includes: acquiring raw blood; performing T cell sorting for the raw blood; performing amplification culture for the sorted T cells; performing CAR transfection for the amplification cultured T cells; performing re-amplification culture for the CAR transfected T cells; treating another batch of T cells during the amplification culture and the re-amplification culture; acquiring the cultured CAR-T cells.

### CROSS REFERENCE TO RELATED APPLICATION

The present application is a continuation application of PCT/CN2018/096105, filed on Jul. 18, 2018, which claims priority to Chinese patent application No. CN201810666385.3 filed on Jun. 26, 2018, the contents of which are all hereby incorporated by reference.

### FIELD OF THE INVENTION

The present application relates to a cell culture method, and more particularly to a fully automatic cell culture method and system thereof based on mechanical arm.

### BACKGROUND OF THE INVENTION

Cell therapy is leading the future medical revolution. Cell therapy has become the fourth largest medical method after protein drugs, chemical drugs and medical equipment. In cell therapy, the cell culture process is the foundation for supporting cell therapy; however, the current traditional cell culture process has the following deficiencies:

For local process steps of the whole production process, the existing manual semi-automated equipment on the market can only produce for a single patient in the same time period, which has limited benefit to realize the industrialized cell production scale and effectively reduce the production cost. Traditional methods of artificially culturing cells have disadvantages such as low efficiency, high probability of contamination, high error rate, and difficulty in quality monitoring, and due to the different experience, techniques and habits of the operators, the status of the cultured cells is often inconsistent, making the culture process has poor reproducibility, stability, and uniformity, thereby affecting the quality of cells. At present, the most reliable way is to use automatic system to replace artificial culture, which has become the trend of current industry. At the same time that the cell application demand is experiencing explosive growth, more stringent requirements are put forward in terms of improving the efficiency of cell production and preparation, effectively reducing the production and production cost and unifying the quality standard of production and preparation, to meet the huge market capacity and the diversified demand of precision medicine.

At present, there are some automated single-unit devices on the market; however, it can only complete a certain step or a certain operation of the cell culture process and it still need manual work to connect the entire production process, and it is unable to realize full process automation. At present, the operating environment of cell culture is usually a partial open A level in the C-level background, or a partially open A level in the B-level background. This traditional design has many disadvantages in regards to GMP, and the hardware investment of the plant and the maintenance cost in the later stages is high, causing the cost of cell therapy remains high.

Therefore, it is necessary to design a new cell culture method to realize the fully automatic culture of cell, and in the process of culturing a batch of cells, another batch of cells could be intermittently cultured at the same time, improving the efficiency of cell culture and saving the preparation cost.

### SUMMARY OF THE INVENTION

The present application provides a fully automatic cell culture method and system thereof based on mechanical arm.

In order to achieve the above object, the present application adopts the following technical solution: a fully automatic cell culture method based on mechanical arm, comprising:

acquiring raw blood;

performing T cell sorting for the raw blood;

performing amplification culture for the sorted T cells;

performing CAR transfection for the amplification cultured T cells;

performing re-amplification culture for the CAR transfected T cells;

treating another batch of T cells during the amplification culture and the re-amplification culture;

acquiring the cultured CAR-T cells.

A further technical solution is: the step of treating another batch of T cells during the amplification culture and the re-amplification culture comprises the following steps:

sterilizing a liquid storage tank;

culturing another batch of T cells.

The further technical solution is: the step of culturing another batch of T cells comprises: performing T cell sorting, and/or performing amplification culture for the sorted T cells, and/or, performing CAR transfection for the amplification cultured T cells, and/or, performing re-amplification culture for the CAR transfected T cells.

The further technical solution is: the step of performing CAR transfection for the amplification cultured T cells comprises the following steps:

adding virus into an empty bag to culture for a plurality of hours;

transferring blood into the bag and culturing for a plurality of hours;

washing the substance in the bag and transfer the substance to a new bag.

The present application further provides a fully automatic cell culture system based on mechanical arm, comprising: a sorting unit, a culture unit, a centrifugal transfection unit and an acquiring unit;

wherein, the sorting unit, for performing T cell sorting for raw blood;

the culture unit, for performing amplification culture for the sorted T cells and performing re-amplification culture for CAR transfected T cells;

the centrifugal transfection unit, for performing CAR transfection for the amplification cultured T cells;

the acquiring unit, for acquiring the cultured CAR-T cells.

The further technical solution is: the system further comprises a transfer unit for transferring cells among the sorting unit, the culture unit, the centrifugal transfection unit and the acquiring unit, and the transfer unit comprises a six degree of freedom GMP-compliant robot, a clamping fixture and a dustproof linear guide, and the clamping fixture is connected to the six degree of freedom GMP-compliant robot, and the six degree of freedom GMP-compliant robot is disposed on the dustproof linear guide.

The further technical solution is: the culture unit comprises a CO₂ incubator and a culture bag storage module.

The further technical solution is: the centrifugal transfection unit comprises a centrifuge.

The further technical solution is: the acquiring unit comprises a detecting module, a discharging module, and a waste storage module;

the detecting module, for collecting and detecting basic quality data of the acquired cells;

the discharging module, for discharging a finished product;

the waste storage module, for storing the used consumables and reagent.

The further technical solution is: the system further comprises an environment control unit;

the environment control unit, for controlling the sorting unit, the culture unit, the centrifugal transfection unit and the acquiring unit being in a GMP-compliant sterile environment.

Compared with prior art, the beneficial effects of the present application are: the fully automatic cell culture method based on mechanical arm of the present application uses a mechanical arm to transfer the cells among various stages of cell culture, and could treat another batch of T cells during the amplification culture and re-amplification culture, and could interpolate the culture of another batch of cells into the process of culturing one batch of cells, to improve the efficiency of cell culture and save the preparation cost. The centrifugal transfection and collecting the cultured cells are also automatically performed, thereby realizing fully automatic cell culture and improving the efficiency of cell culture.

The present application is further described below in conjunction with the accompanying drawings and the embodiments.

### BRIEF DESCRIPTION OF THE DRAWINGS

FIG. 1 is a flow chart 1 of a fully automatic cell culture method based on mechanical arm according to an embodiment of the present application;

FIG. 2 is a flow chart 2 of a fully automatic cell culture method based on mechanical arm according to an embodiment of the present application;

FIG. 3 is a specific flow chart of treating another batch of T cells during amplification culture and re-amplification culture according to an embodiment of the present application;

FIG. 4 is a specific flow chart of CAR transfection of amplification cultured T cells according to an embodiment of the present application;

FIG. 5 is a structural block diagram of a fully automatic cell culture system based on mechanical arm according to an embodiment of the present application;

FIG. 6 is a structural block diagram of an acquiring unit according to an embodiment of the present application.

### DESCRIPTION OF THE EMBODIMENTS

In order to more fully understand the technical content of the present application, the technical solutions of the present application are further described and illustrated below in conjunction with the embodiments, but are not limited thereto.

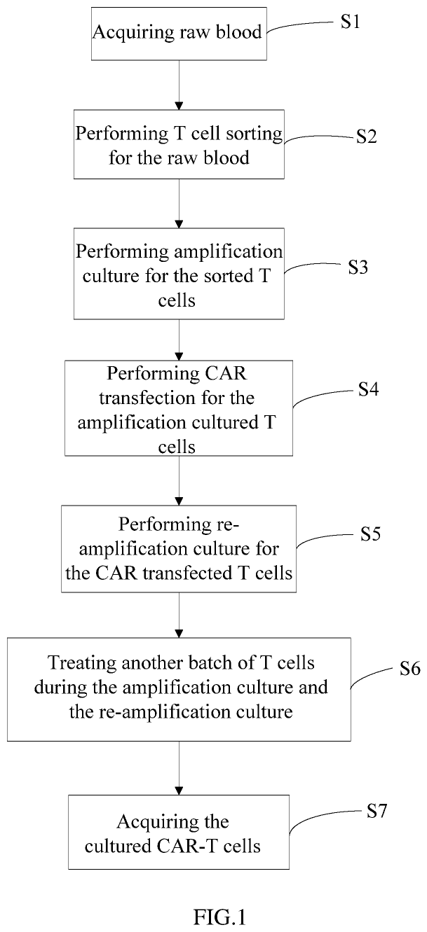

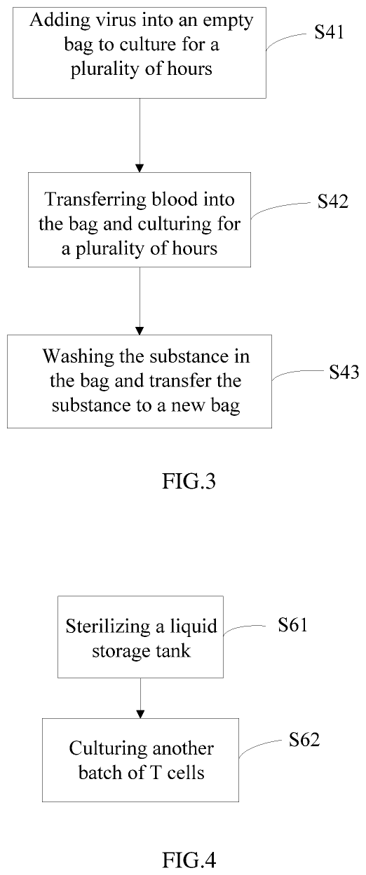

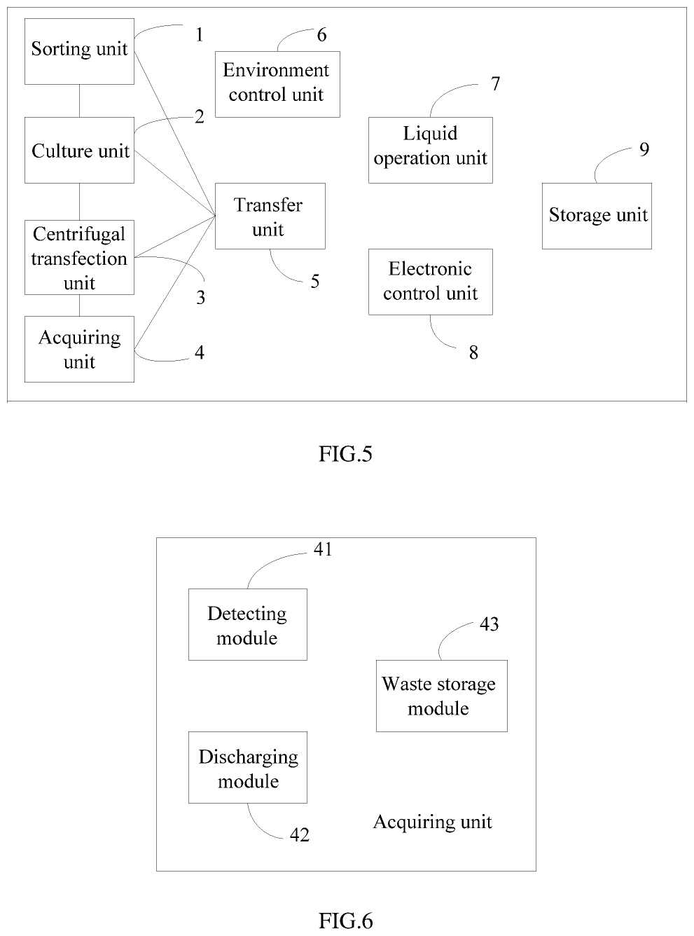

As shown in FIG. 1 to FIG. 6, a fully automatic cell culture method based on mechanical arm according to the present embodiment could be applied to culture various cells such as red blood cells, to realize fully automatic cell culture, and in the process of culturing a cell, another cell could be intermittently cultured, improving the efficiency of cell culture and saving the preparation cost.

As shown in FIG. 1, the present embodiment provides a fully automatic cell culture method based on mechanical arm, comprising:

S1, acquiring raw blood;

S2, performing T cell sorting for the raw blood;

S3, performing amplification culture for the sorted T cells;

S4, performing CAR transfection for the amplification cultured T cells;

S5, performing re-amplification culture for the CAR transfected T cells;

S6, treating another batch of T cells during the amplification culture and the re-amplification culture;

S7, acquiring the cultured CAR-T cells.

In the steps S6 to S7, specifically, using a mechanical arm to transfer the raw blood and the cell among various links.

For the above step S1, specifically, acquiring the raw blood that needs to culture cells, for example, the patient's raw blood, etc.

For the above step S2, performing T cell sorting for the raw blood, specifically, separating batches of cells by density gradient centrifugation, washing the separated cells, sampling and counting the washed cells, adding OKT3, adjusting density, performing cell inoculation to complete the cell sorting.

Further, in some embodiments, for the above step S4, performing CAR transfection for the amplification cultured T cells comprises the following specific steps:

S41, adding virus into an empty bag to culture for a plurality of hours;

S42, transferring blood into the bag and culturing for a plurality of hours;

S43, washing the substance in the bag and transfer the substance to a new bag.

For the above steps S3 to S4, it need to: sample the inoculated cells to calculate the quantity and the survival rate, photograph and save; package separately by the quantity; add IL-2 and culture medium; test the cells which are in the process of the culture preparation.

For the above step S6, treating another batch of T cells during the amplification culture and the re-amplification culture comprises the following specific steps:

S61, sterilizing a liquid storage tank;

S62, culturing another batch of T cells.

Set the liquid storage tank to be two separate spaces, or set the liquid storage tank to be two liquid storage tanks with the same function, and the two tanks are mutual independent, which could be sterilized separately without interference from each other, and the two tanks could realize quick switch among different batches of cell culture to ensure that there is no interference with each other and cross contamination among different batches; realize simultaneous culture of multi-batch, multi-patient cell, greatly improving production efficiency and reducing cost under the premise of meeting the GMP requirement.

For the above step S62, the step of culturing another batch of T cells comprises performing T cell sorting, and/or performing amplification culture for the sorted T cells, and/or, performing CAR transfection for the amplification cultured T cells, and/or, performing re-amplification culture for the CAR transfected T cells.

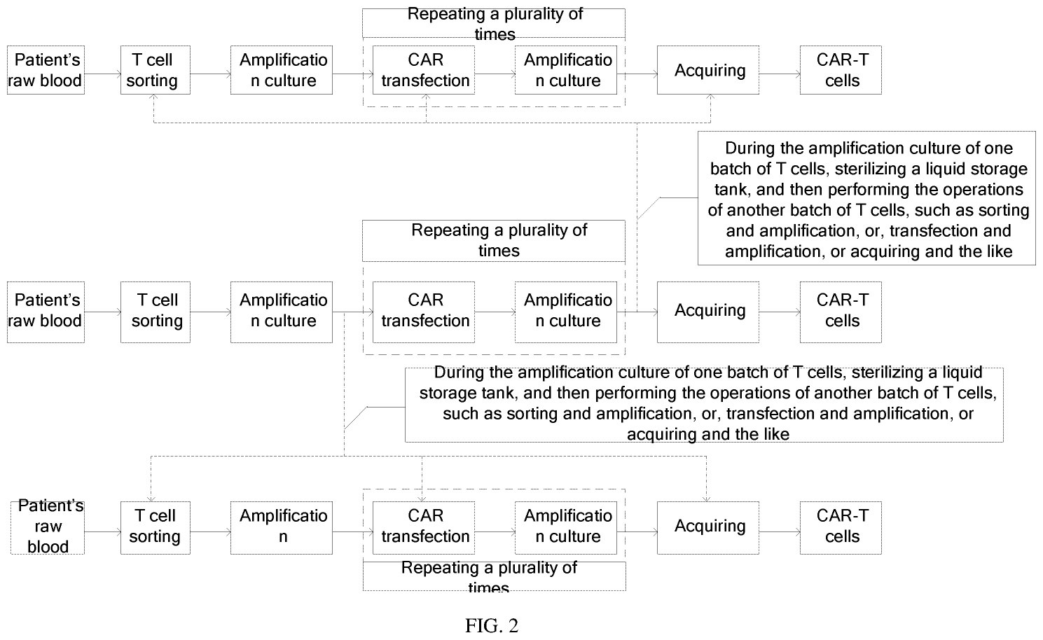

As shown in FIG. 2, after the step S62, the present batch of T cells are transferred to the culture environment of the next step with the assistance of the mechanical arm, thereby performing rapid culture, that is, for the cell culture of each batch, the culture process of the above steps S1 to S7 must be completed, and the difference among each batches is that which batch's cell amplification culture or re-amplification culture process the current cell is interspersed with.

For the above step S7, specifically: acquiring cells in a new culture bag; washing the cells; sampling and counting the washed cells; performing factory test for the counted cells; freezing and storing the cells that meet the factory test, and storing them in the sampling tube.

Being designed based on a high degree of freedom robot (mechanical arm) and integrated with a general cell preparation/quality inspection equipment, the robot (mechanical arm) simulates manual work to perform various process operations of various cell cultures, to complete the process such as cell separation and sorting, infection, liquid operation, culture, collection, cryopreservation and encapsulation, and cooperates with the quality inspection module 41 in the production process, to automatically complete the detection of relevant central control items. Being integrated with an environmental control module, each compartment is independent and sealed to form a Class A space that is completely isolated from the background environment and the equipment could be installed and operated in the lowest cleanliness class: Class D environment. A system corresponding to the method could be flexibly installed in a hospital, clinic, biological service company, and pharmaceutical enterprise, which has high adaptability to the environment. The system could also integrate a space sterilization system to meet the sterilization requirements of different production processes, and the sterilization process could be repeatedly verified and meet the GMP requirement.

The mechanical arm automatically transfers the cells among various culture processes to achieve automatic culture of the cells. Under the premise of ensuring that the cost such as equipment procurement, plant infrastructure construction and consumable procurement is not higher than traditional manual method, the method could greatly reduce the operating cost; exclude the interference caused by human and environmental factors in the production process, and effectively improve the stability and reproducibility of the cell preparation process, thereby effectively improving the uniformity and stability of the product quality; effectively avoid human operation error, and reduce the leakage risk of intellectual property of the core preparation which is caused by the turnover, and greatly reduce the high cost of repeat personnel training; interpolate the culture of another batch of cells into the amplification culture step in the cell culture process, and simultaneously prepare multi-batch, greatly improving the cell preparation efficiency and saving the preparation cost, thereby accelerating the promotion and popularization of cell therapy, and benefiting the majority of patients.

The above fully automatic cell culture method based on mechanical arm uses a mechanical arm to transfer the cells among various stages of cell culture, and could treat another batch of T cells during the amplification culture and re-amplification culture, and could interpolate the culture of another batch of cells into the process of culturing one batch of cells, to improve the efficiency of cell culture and save the preparation cost. The centrifugal transfection and collecting the cultured cells are also automatically performed, thereby realizing fully automatic cell culture and improving the efficiency of cell culture.

As shown in FIG. 5, the present embodiment also provides a fully automatic cell culture system based on mechanical arm which comprises a sorting unit 1, a culture unit 2, a centrifugal transfection unit 3 and an acquiring unit 4.

Wherein, the sorting unit 1, for performing T cell sorting for raw blood;

the culture unit 2, for performing amplification culture for the sorted T cells and performing re-amplification culture for CAR transfected T cells;

the centrifugal transfection unit 3, for performing CAR transfection for the amplification cultured T cells;

the acquiring unit 4, for acquiring the cultured CAR-T cells.

The above system further comprises a transfer unit 5 for transferring cells among the sorting unit 1, the culture unit 2, the centrifugal transfection unit 3 and the acquiring unit 4, and the transfer unit 5 comprises a six degree of freedom GMP-compliant robot, a clamping fixture and a dustproof linear guide, and the clamping fixture is connected to the six degree of freedom GMP-compliant robot, and the six degree of freedom GMP-compliant robot is disposed on the dustproof linear guide.

The transfer unit 5 performs the transportation of patient cell samples, preparation consumable and the like among different working modules, and participates in process operations such as adding liquid.

Being designed based on a high degree of freedom robot (mechanical arm) and integrated with a general cell preparation/quality inspection equipment, the robot (mechanical arm) simulates manual work to perform various process operations of various cell cultures, to complete the process such as cell separation and sorting, infection, liquid operation, culture, collection, cryopreservation and encapsulation, and cooperates with the quality inspection module 41 in the production process, to automatically complete the detection of relevant central control items. Being integrated with an environmental control module, each compartment is independent and sealed to form a Class A space that is completely isolated from the background environment and the equipment could be installed and operated in the lowest cleanliness class: Class D environment. A system corresponding to the method could be flexibly installed in a hospital, clinic, biological service company, and pharmaceutical enterprise, which has high adaptability to the environment. The system could also integrate a space sterilization system to meet the sterilization requirements of different production processes, and the sterilization process could be repeatedly verified and meet the GMP requirement.

The mechanical arm automatically transfers the cells among various culture processes to achieve automatic culture of the cells. Under the premise of ensuring that the cost such as equipment procurement, plant infrastructure construction and consumable procurement is not higher than traditional manual method, the method could greatly reduce the operating cost; exclude the interference caused by human and environmental factors in the production process, and effectively improve the stability and reproducibility of the cell preparation process, thereby effectively improving the uniformity and stability of the product quality; effectively avoid human operation error, and reduce the leakage risk of intellectual property of the core preparation which is caused by the turnover, and greatly reduce the high cost of repeat personnel training; interpolate the culture of another batch of cells into the amplification culture step in the cell culture process, and simultaneously prepare multi-batch, greatly improving the cell preparation efficiency and saving the preparation cost, thereby accelerating the promotion and popularization of cell therapy, and benefiting the majority of patients.

Further, the culture unit 2 comprises a CO₂ incubator and a culture bag storage module. When the cells are cultured, they are cultured in the CO₂ incubator.

Further, the centrifugal transfection unit 3 comprises a centrifuge for performing operations such as virus (centrifugation) transfection, cell acquiring and the like.

Further, in some embodiments, the acquiring unit 4 comprises a detecting module 41, a discharging module 42, and a waste storage module 43.

The detecting module 41, for collecting and detecting basic quality data of the acquired cells. It comprises an automatic inverted microscope, a flow cytometer, and an optical cytometer, which performs the collection and detection of the basic quality data during the preparation process, such as the total number of cells, the cell proliferation rate, cell viability, transfection efficiency, and cell subsets.

The discharging module 42, for discharging a finished product.

The waste storage module 43, for storing the used consumables and reagent.

In addition, the system further comprises an environment control unit 6.

The environment control unit 6, for controlling the sorting unit 1, the culture unit 2, the centrifugal transfection unit 3 and the acquiring unit 4 being in a GMP-compliant sterile environment.

The environment control unit 6 comprises laminar flow modules independently set up for each compartment and an H₂O₂ sterilization module. The system integrates the environment control, and each compartment is independently sealed to form a Class A dynamic laminar flow space, and the equipment could be installed and operate in a Class D environment. The system could be flexibly installed in a hospital, clinic, biological service company, and pharmaceutical enterprise, which has high adaptability to the environment. The system could also integrate a sterilization and disinfection system to meet the sterilization and disinfection requirements of different production processes.

In addition, as a preferred embodiment, the system further comprises a liquid operation unit 7, specifically, the liquid operation unit 7 comprises a bagging/bottling liquid operation module, a large-capacity reagent solution operation module, a small-capacity high-precision liquid operation module, an opening/closing bottle module and a magnet operation module, and the liquid operation unit 7 is for performing liquid operation of different capacity and different precision requirements in various process steps.

The system further comprises a refrigerator group which comprises three temperature grades of −80° C., −20° C., and 4° C. to provide an environment for cryopreservation and the like.

The system further comprises an electronic control unit 8, specifically, the electronic control unit 8 comprises a high-performance industrial computing server, a PLC control module, various types of sensor networks and a power module, etc., to provide power for each unit.

In addition, the system further comprises a storage unit 9 for temporary storage of materials used in the production process in the system. The system further comprises a data collection unit, a quality management unit and a remote customer service unit and the like, adapting to the traditional production center's production mode, and creating a new distributed cell preparation production model at the same time, closing to the end-users and providing direct service, and having high adaptability to different production models and business models.

The above fully automatic cell culture system based on mechanical arm uses a mechanical arm to transfer the cells among various stages of cell culture, and could treat another batch of T cells during the amplification culture and re-amplification culture, and could interpolate the culture of another batch of cells into the process of culturing one batch of cells, to improve the efficiency of cell culture and save the preparation cost. The centrifugal transfection and collecting the cultured cells are also automatically performed, thereby realizing fully automatic cell culture and improving the efficiency of cell culture.

The technical content of the present application is further described by way of embodiments only, so as to be easily understood by the reader, but the embodiment of the present application is not limited thereto, and any technical extension or re-creation made according to the present application should all fall into the protection of the present application. The protection scope of the present application is subject to the claims.

What is claimed is:

1. A fully automatic cell culture method based on mechanical arm, comprising:
acquiring raw blood;
performing T cell sorting for the raw blood;
performing amplification culture for the sorted T cells;
performing CAR transfection for the amplification cultured T cells;
performing re-amplification culture for the CAR transfected T cells;
treating another batch of T cells during the amplification culture and the re-amplification culture;
acquiring the cultured CAR-T cells.

2. The fully automatic cell culture method based on mechanical arm according to claim 1, wherein the step of treating another batch of T cells during the amplification culture and the re-amplification culture comprises the following steps:
sterilizing a liquid storage tank;
culturing another batch of T cells.

3. The fully automatic cell culture method based on mechanical arm according to claim 2, wherein the step of culturing another batch of T cells comprises: performing T cell sorting, and/or performing amplification culture for the sorted T cells, and/or, performing CAR transfection for the amplification cultured T cells, and/or, performing re-amplification culture for the CAR transfected T cells.

4. The fully automatic cell culture method based on mechanical arm according to claim 3, wherein the step of performing CAR transfection for the amplification cultured T cells comprises the following steps:
adding virus into an empty bag to culture for a plurality of hours;
transferring blood into the bag and culturing for a plurality of hours;
washing the substance in the bag and transfer the substance to a new bag.

5. A fully automatic cell culture system based on mechanical arm, comprising: a sorting unit, a culture unit, a centrifugal transfection unit and an acquiring unit;
wherein, the sorting unit, for performing T cell sorting for raw blood;
the culture unit, for performing amplification culture for the sorted T cells and performing re-amplification culture for CAR transfected T cells;
the centrifugal transfection unit, for performing CAR transfection for the amplification cultured T cells;
the acquiring unit, for acquiring the cultured CAR-T cells.

6. The fully automatic cell culture system based on mechanical arm according to claim 5, wherein the system further comprises a transfer unit for transferring cells among the sorting unit, the culture unit, the centrifugal transfection unit and the acquiring unit, and the transfer unit comprises a six degree of freedom GMP-compliant robot, a clamping fixture and a dustproof linear guide, and the clamping fixture is connected to the six degree of freedom GMP-compliant robot, and the six degree of freedom GMP-compliant robot is disposed on the dustproof linear guide.

7. The fully automatic cell culture system based on mechanical arm according to claim 6, wherein the culture unit comprises a CO₂ incubator and a culture bag storage module.

8. The fully automatic cell culture system based on mechanical arm according to claim 7, wherein the centrifugal transfection unit comprises a centrifuge.

9. The fully automatic cell culture system based on mechanical arm according to claim 8, wherein the acquiring unit comprises a detecting module, a discharging module, and a waste storage module;
the detecting module, for collecting and detecting basic quality data of the acquired cells;
the discharging module, for discharging a finished product;
the waste storage module, for storing the used consumables and reagent.

10. The fully automatic cell culture system based on mechanical arm according to claim 9, wherein the system further comprises an environment control unit;
the environment control unit, for controlling the sorting unit, the culture unit, the centrifugal transfection unit and the acquiring unit being in a GMP-compliant sterile environment.
